(19) United States
(12) Patent Application Publication
(10) Pub. No.: US 2024/0310498 A1
(43) Pub. Date: Sep. 19, 2024

(54) RECHARGEABLE PORTABLE ULTRASOUND INSTRUMENT, POWER SOURCE CONTROL METHOD, AND PROGRAM

(71) Applicant: KONICA MINOLTA, INC., Tokyo (JP)

(72) Inventor: Morio NISHIGAKI, Fujisawa-shi (JP)

(73) Assignee: KONICA MINOLTA, INC., Tokyo (JP)

(21) Appl. No.: 18/581,437

(22) Filed: Feb. 20, 2024

(30) Foreign Application Priority Data
Mar. 16, 2023 (JP) 2023-041871

(51) Int. Cl. G01S 7/521; A61B 8/00; H02J 7/34

(52) U.S. Cl. CPC G01S 7/521; A61B 8/4427; H02J 7/345; A61B 2560/0214; A61B 2560/0431; H02J 2207/50

(57) ABSTRACT

A rechargeable portable ultrasound instrument for transmitting an ultrasound wave to a subject and receiving the ultrasound wave from the subject for ultrasound diagnosis includes: at least two chargers different in type, the chargers including a supercapacitor and a battery; and a power source controller that controls a charge operation to each of the two chargers and a power supply operation from each of the chargers to an instrument body of the rechargeable portable ultrasound instrument, wherein the power source controller controls the charge operation and the power supply operation in accordance with the type of each of the chargers as a charge target or a power supply target.

The entire disclosure of Japanese patent Application No. 2023-041871, filed on Mar. 16, 2023, is incorporated herein by reference in its entirety.

### BACKGROUND

### Technological Field

The present invention relates to a rechargeable portable ultrasound instrument, a power source control method, and a program.

### Description of the Related art

Conventionally, as one of medical diagnostic imaging apparatuses. known has been a diagnostic ultrasound apparatus that transmits an ultrasound wave toward a subject. receives a reflected wave thereof. and performs predetermined signal processing on a reception signal to visualize the shape, property, or dynamics inside the subject as an ultrasound image (see. for example. JP 2002-85405 A and JP 2019-198457 A.) Such a diagnostic ultrasound apparatus can acquire an ultrasound image resulting from applying an ultrasound probe to the body surface of the subject. Thus, the diagnostic ultrasound apparatus is safe and the burden on the subject is small.

JP 2002-85405 A discloses a small handheld diagnostic ultrasound apparatus that can be carried and used. Further, JP 2019-198457 A discloses a wireless ultrasound probe that is wirelessly connectable to a diagnostic ultrasound apparatus. Such handheld diagnostic ultrasound apparatus and wireless ultrasound probe typically incorporate a rechargeable battery power source. Hereinafter, the handheld diagnostic ultrasound apparatus and the wireless ultrasound probe are collectively referred to as a “rechargeable portable ultrasound instrument”.

A rechargeable portable ultrasound instrument is typically connected to a charger such as a cradle to be charged. Depending on the residual battery level. the charging time ranges from several tens of minutes to several hours. Because an available place is limited during charging. it is assumed that the rechargeable portable ultrasound instrument is fully charged during a closed hour such as nighttime.

However, due to use of the rechargeable portable ultrasound instrument without charging for a long time, its residual battery level may be insufficient. It is conceivable to rapidly charge the battery power source by supplying large power from an external power source to the battery power source. However, there is a limit to shortening the charging time for a lithium ion battery as a mainstream of the current battery power sources.

### SUMMARY

An object of the present invention is to provide a rechargeable portable ultrasound instrument, a power source control method, and a program that enable shortening of a time restricting from use due to charging and enable improvement in convenience of a diagnostic ultrasound apparatus.

To achieve the abovementioned object, according to an aspect of the present invention, a rechargeable portable ultrasound instrument for transmitting an ultrasound wave to a subject and receiving the ultrasound wave from the subject for ultrasound diagnosis, reflecting one aspect of the present invention comprises: at least two chargers different in type, the chargers including a supercapacitor and a battery; and a power source controller that controls a charge operation to each of the two chargers and a power supply operation from each of the chargers to an instrument body of the rechargeable portable ultrasound instrument, wherein the power source controller controls the charge operation and the power supply operation in accordance with the type of each of the chargers as a charge target or a power supply target.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is an illustration of the exterior appearance of a diagnostic ultrasound apparatus according to an embodiment;

FIG. 2 is a block diagram of main parts of a control system of the diagnostic ultrasound apparatus;

FIGS. 3A to 3C are diagrams of an exemplary electric circuit as a constituent of a power source controller according to a first embodiment;

FIG. 4 is a flowchart of exemplary power source control processing according to the first embodiment;

FIGS. 5A to 5C are diagrams of an exemplary electric circuit as a constituent of a power source controller according to a second embodiment;

FIG. 6 is a flowchart of exemplary power source control processing according to the second embodiment;

FIGS. 7A and 7B are explanatory diagrams of assist drive; and

FIGS. 8A and 8B are diagrams of an exemplary electric circuit as a constituent of a supercapacitor suitable for assist drive.

### DETAILED DESCRIPTION OF EMBODIMENTS

Hereinafter, one or more embodiments of the present invention will be described with reference to the drawings. However, the scope of the invention is not limited to the disclosed embodiments.

### First Embodiment

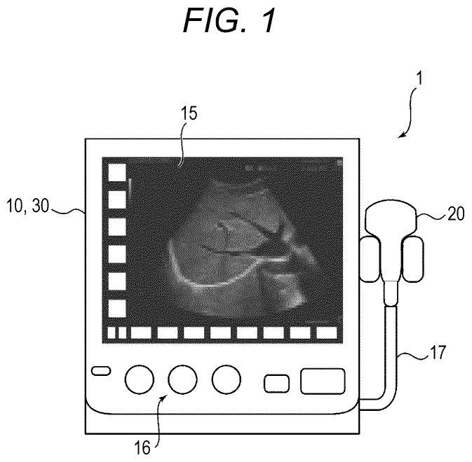

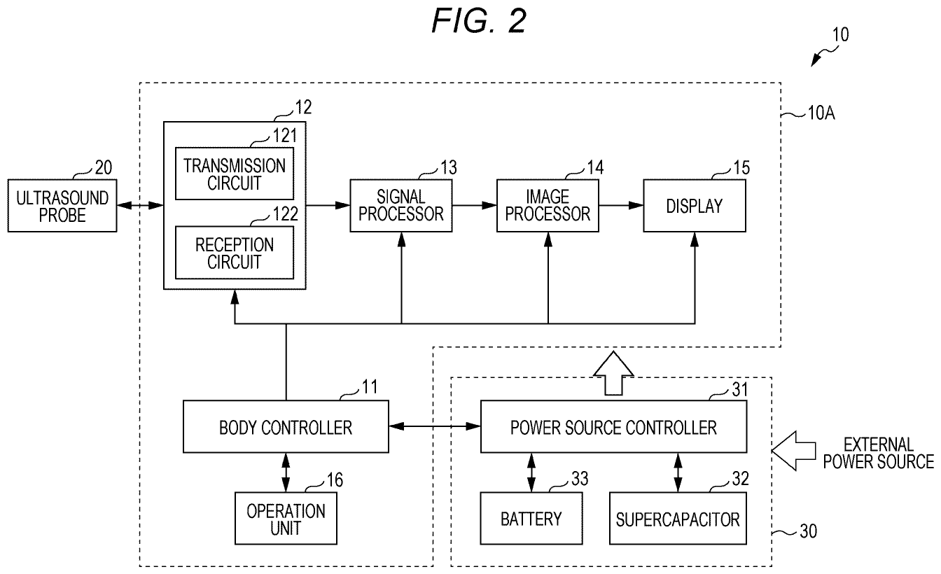

FIG. 1 is an illustration of the exterior appearance of a diagnostic ultrasound system 1 according to an embodiment of the present invention. FIG. 2 is a block diagram of main parts of a control system of the diagnostic ultrasound system 1. In the present embodiment. a rechargeable portable ultrasound instrument according to the present invention is applied to a diagnostic ultrasound apparatus 10.

The diagnostic ultrasound system 1 is used to visualize the shape, property, or dynamics in a subject as an ultrasound image and perform diagnostic imaging.

As illustrated in FIG. 1. the diagnostic ultrasound system 1 includes the diagnostic ultrasound apparatus 10 and an ultrasound probe 20. The diagnostic ultrasound apparatus 10 and the ultrasound probe 20 are connected so as to be communicable in a wired manner through a cable 17, for example. Note that the diagnostic ultrasound apparatus 10 and the ultrasound probe 20 may be connected so as to be wirelessly communicable.

The ultrasound probe 20 transmits an ultrasound wave to the subject and receives an ultrasound echo (reflected ultrasound wave) reflected on the subject. The ultrasound probe 20 converts the ultrasound echo into a reception signal to transmit the reception signal to the diagnostic ultrasound apparatus 10.

As the ultrasound probe 20, applicable is any electronic scanning transducer such as a convex transducer, a linear transducer, or a sector transducer, or a mechanical scanning transducer such as a mechanical sector transducer. The ultrasound probe 20 may have a puncture needle guide to which a puncture needle is attached and that guides the direction of insertion.

For example, the ultrasound probe 20 includes an acoustic lens, an acoustic matching layer, an oscillator array, and a backing material (all not illustrated) in order from the ultrasound wave transmission and reception side (surface side of the probe head). Note that a protective layer may be disposed on the surface (ultrasound transmission and reception face) of the acoustic lens.

The acoustic lens is a lens that converges an ultrasound wave in a slice direction (a direction orthogonal to a scanning direction in which a plurality of oscillators is arrayed). For example, in a case where a material slower in sound speed than a living body is used for the acoustic lens, the acoustic lens typically has a semi-cylindrical shape in which a central portion in the slice direction swells. The acoustic matching layer is an intermediate substance for efficiently allowing an ultrasound wave to enter the subject, and matches acoustic impedance between such an oscillator as described above and the subject. The oscillator array includes, for example, a plurality of strip-shaped oscillators arranged in a single line in the scanning direction. The backing material attenuates unnecessary oscillation generated at the oscillator array.

The diagnostic ultrasound apparatus 10 uses the reception signal from the ultrasound probe 20 to visualize the shape, property, or dynamics in the subject as an ultrasound image (B-mode image).

The diagnostic ultrasound apparatus 10 includes a body controller 11, a transmitter/receiver 12, a signal processor 13, an image processor 14, a display 15, an operation unit 16, and a body power source 30. The apparatus body 10A including the body controller 11, the transmitter/receiver 12, the signal processor 13, the image processor 14, the display 15, and the operation unit 16 is driven by power supply from the body power source 30.

The body controller 11 includes a central processing unit (CPU) as an arithmetic/control device, a read only memory (ROM) and a random access memory (RAM) as a main storage device. for example. The ROM stores a basic program and basic setting data. Further, the ROM stores a diagnostic ultrasound program executed at the time of diagnosis.

The CPU reads a program corresponding to the details of processing from the ROM and develops the program into the RAM. The CPU executes the developed program to centrally control the operation of each functional block (transmitter/receiver 12, signal processor 13, image processor 14, display 15, operation unit 16, and body power source 30) of the diagnostic ultrasound apparatus 10.

The function of each functional block is achieved by, for example, cooperation between each piece of hardware serving as the corresponding functional block and the body controller 11. Note that part or all of the functions of each functional block may be achieved resulting from execution of a program by the body controller 11.

The transmitter/receiver 12 includes a transmission circuit 121 and a reception circuit 122.

In accordance with an instruction from the body controller 11, the transmission circuit 121 generates a transmission signal (drive signal), and outputs the transmission signal to the ultrasound probe 20. Although not illustrated, the transmission circuit 121 includes, for example, a clock generation circuit, a delay circuit, a pulse generation circuit, and a pulse width setting unit.

The clock generation circuit generates a clock signal for determination of the transmission timing and the transmission frequency of a pulse signal. The delay circuit counts such clock signals as described above on the basis of a trigger signal from the body controller 11, and generates a transmission timing signal determined for each oscillator. On the basis of the transmission timing signal and the clock signals, the pulse generation circuit generates a bi-polar rectangular wave pulse having a voltage amplitude of a waveform set in advance by the pulse width setting unit. Such rectangular wave pulses generated in the pulse generation circuit are distributed to respective different wiring paths for the oscillators of the ultrasound probe 20, and are output from the oscillators.

In accordance with an instruction from the body controller 11, the reception circuit 122 receives a reception signal from the ultrasound probe 20, and outputs the reception signal to the signal processor 13. Although not illustrated, the reception circuit 122 includes, for example, an amplifier, an A/D conversion circuit, and a phasing addition circuit.

The amplifier amplifies the reception signal corresponding to the ultrasound wave received by each oscillator of the ultrasound probe 20 at a predetermined amplification factor set in advance. The A/D conversion circuit converts the amplified reception signal into digital data at a predetermined sampling frequency. The phasing addition circuit applies a delay time to the A/D-converted reception signal for each wiring path corresponding to the oscillator to adjust the time phase, and adds the time phases (performs phasing addition).

In accordance with an instruction from the body controller 11, the signal processor 13 generates image data indicating the internal state of the subject on the basis of a reception signal obtained by transmission and reception of an ultrasound wave. Examples of the generated image data include a B-mode tomographic image, a blood flow spectrum by Doppler, and a blood flow distribution image.

In accordance with an instruction from the body controller 11, the image processor 14 generates display data on the basis of the B-mode image data from the signal processor 13. Although not illustrated, the image processor 14 includes, for example, a digital scan converter (DSC) that performs coordinate conversion and pixel interpolation corresponding to the type of the ultrasound probe 20.

The signal processor 13 and the image processor 14 each include, for example, dedicated or general-purpose hardware (electronic circuit) corresponding to each piece of processing, such as a digital signal processor (DSP), an application specific integrated circuit (ASIC), or a programmable logic device (PLD), and achieve each function in cooperation with the body controller 11.

The display 15 includes, for example, a liquid crystal display, an organic electro-luminescence (EL) display, or a cathode ray tube (CRT) display. In accordance with an instruction from the body controller 11, the display 15 displays an ultrasound image on the basis of the display data from the image processor 14.

The operation unit 16 receives, for example, a command instructing start of diagnosis or input of information on the subject. The operation unit 16 includes, for example, an operation panel having a plurality of input switches, a keyboard, and a track ball. For example, with the operation unit 16, the user can perform an instruction for execution of diagnostic ultrasound processing. Note that the operation unit 16 may be as a constituent of a touch panel in which the operation unit 16 is provided integrally with the display 15.

Although not illustrated, the diagnostic ultrasound apparatus 10 may include a communication unit that transmits and receives various types of information to and from an external device (e.g., a personal computer or a cloud storage on the Internet) connected to a communication network such as a wired or wireless local area network (LAN). The communication unit includes. for example, various interfaces such as a network interface card (NIC), a modulator-demodulator (MODEM), and a universal serial bus (USB). Further, a communication interface for short-range wireless communication such as near field communication (NFC) or Bluetooth (registered trademark) is applicable to the communication unit.

The body power source 30 supplies drive power to the apparatus body 10A. The body power source 30 includes a power source controller 31, a supercapacitor 32, and a battery 33. That is, the body power source 30 includes two types of chargers including the supercapacitor 32 and the battery 33.

The battery 33 is a charging device capable of repeatedly charging and discharging by a chemical reaction, and includes, for example, a lithium ion battery. For example, the battery 33 in a fully charged state can drive the apparatus body 10A for several hours.

The supercapacitor 32 is, for example, a capacitor having a capacitance of several F or more and excellent in charge-discharge cycle characteristics and rapid charge-discharge characteristics. In a case where the supercapacitor 32 is the same in electric capacity as the battery 33, the charging time is remarkably shorter than that of the battery 33, and if the amount of current for supply is sufficient, charging is completed for about several seconds, for example.

The supercapacitor 32 preferably includes an electric double-layer capacitor. Note that the supercapacitor 32 may include, for example, a pseudo capacitor or a hybrid capacitor.

The supercapacitor 32 has capacitance sufficient to fully charge the battery 33. In this case, after the supercapacitor 32 is fully charged, the battery 33 can be fully charged due to charge transfer from the supercapacitor 32. At the time of completion of charging of the supercapacitor 32, the charging of the diagnostic ultrasound apparatus 10 is substantially completed, and the diagnostic ultrasound apparatus 10 can be removed from the charger and used.

The power source controller 31 controls charge and discharge operations to the supercapacitor 32 and the battery 33, specifically, a charge operation from an external power source (AC power source) to the supercapacitor 32 and the battery 33, and a power supply operation from the supercapacitor 32 and the battery 33 to the apparatus body 10A. For example, due to placement of the diagnostic ultrasound apparatus 10 on the charger, the power source controller 31 is electrically connected to the external power source, and starts a charge operation to the supercapacitor 32 and the battery 33.

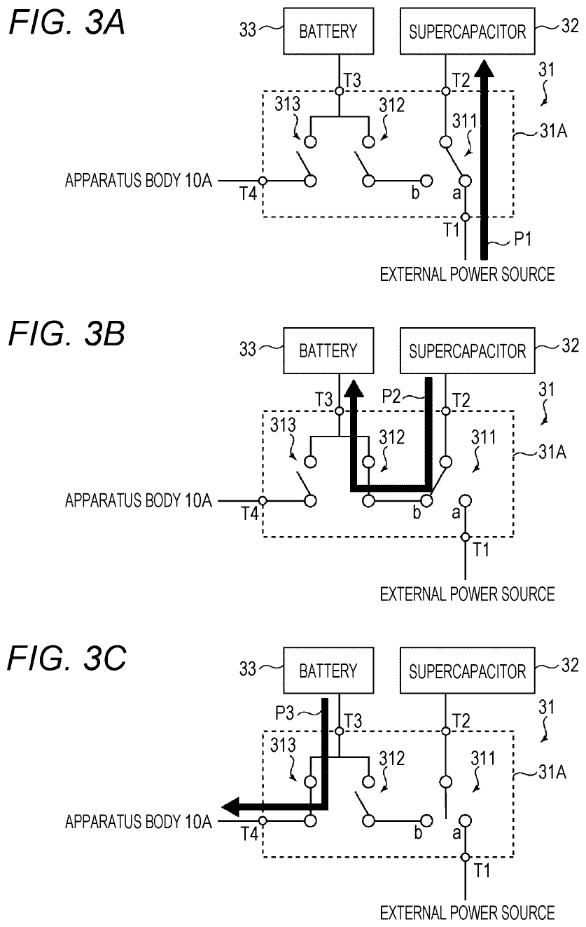

FIGS. 3A to 3C are diagrams of an exemplary electric circuit as a constituent of the power source controller 31 according to the first embodiment.

As illustrated in FIGS. 3A to 3C, the power source controller 31 includes, for example, a switching circuit 31A. The switching circuit 31A includes first to third switches 311 to 313.

Note that the switching circuit 31A illustrated in FIGS. 3A to 3C is an example. Thus, and any electric circuit may be adopted, provided that a function can be achieved as the power source controller 31. Further, because the output voltage of the supercapacitor 32, the input/output voltage of the battery 33, and the input voltage for power supply to the apparatus body 10A are different, a voltage adjustment circuit is provided. The voltage adjustment circuit, however, is omitted in FIGS. 3A to 3C in order to simplify the description of operation.

The first switch 311 can switch a current path, connected to an electrical contact T2 with the supercapacitor 32, to a first current path P1 or a second current path P2. The first current path P1 electrically connects an electrical contact T1 with the external power source and the electrical contact T2 with the supercapacitor 32 (see FIG. 3A). The second current path P2 electrically connects the electrical contact T2 with the supercapacitor 32 and an electrical contact T3 with the battery 33.

The second switch 312 and the third switch 313 can switch a current path, connected to the electrical contact T3 with the battery 33, to the second current path P2 or a third current path P3. The third current path P3 electrically connects the electrical contact T3 with the battery 33 and an electrical contact T4 with the apparatus body 10A (sec FIG. 3C).

As illustrated in FIG. 3A, in response to connection of the first switch 311 to the a-side, the first current path P1 is enabled and the supercapacitor 32 can be charged from the external power source.

As illustrated in FIG. 3B, when the second switch 312 is turned on in response to connection of the first switch 311 to the b-side, the second current path P2 is enabled and charging (power transfer) from the supercapacitor 32 to the battery 33 can be made.

As illustrated in FIG. 3C, when the second switch 312 is turned off and the third switch 313 is turned on, the third current path P3 is enabled and power can be supplied from the battery 33 to the apparatus body 10A.

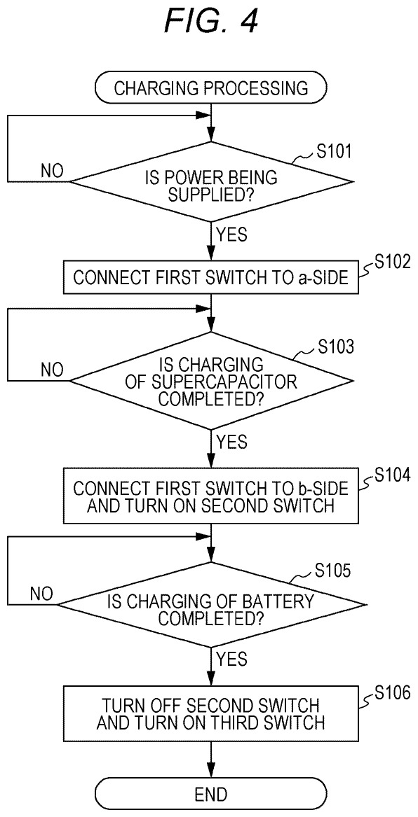

In cooperation with the body controller 11, the power source controller 31 performs charging processing and power supply processing to the supercapacitor 32 and the battery 33. For example, the body controller 11 and the power source controller 31 cooperate to execute a power source control program stored in the ROM of the body controller 11, thereby achieving charging and discharging processing (power source control processing) to the supercapacitor 32 and the battery 33. Specifically, the charging processing to the supercapacitor 32 and the battery 33 is performed in accordance with the flowchart illustrated in FIG. 4.

FIG. 4 is a flowchart of exemplary power source control processing (charging processing) according to the first embodiment. This processing is achieved, for example, by the power source controller 31 executing the power source control program as the diagnostic ultrasound apparatus 10 is placed on the charger and the body power source 30 and the external power source are electrically connected.

In step S101 of FIG. 4, the power source controller 31 determines whether or not power is being supplied from the external power source. If power is being supplied from the external power source (“YES” in step S101), the processing proceeds to step S102. If no power is being supplied from the external power source (“NO” in step S102), the processing of step S101 is repeated. For example, on the basis of a change in the voltage of the electrical contact T1 with the external power source in the switching circuit 31A, a determination can be made as to whether or not power is being supplied from the external power source.

In step S102, the power source controller 31 connects the first switch 311 to the a-side. The first current path P1 is enabled, and charging of the supercapacitor 32 from the external power source starts. The charging of the supercapacitor 32 is completed in an extremely short time of, for example, about several seconds.

In step S103. the power source controller 31 determines whether or not the charging of the supercapacitor 32 is completed. If the charging of the supercapacitor 32 is completed (“YES” in step S103), the processing proceeds to step S104. If the charging of the supercapacitor 32 is not completed (“NO” in step S103), the processing of step S103 is repeated. For example, on the basis of the measurement result of the voltage across the terminals of the supercapacitor 32 or the charging time, a determination can be made as to whether or not the charging of the supercapacitor 32 is completed.

In step S104, the power source controller 31 connects the first switch 311 to the b-side and turns on the second switch 312. The first current path P1 is cut off and the second current path P2 is enabled, so that charging (power transfer) from the supercapacitor 32 to the battery 33 starts.

The charging of the battery 33 is performed at a low speed in accordance with the charging characteristics of the battery 33, and is completed in several hours, for example. The battery 33 is charged by the supercapacitor 32, thereby eliminating connection of the diagnostic ultrasound apparatus 10 to the external power source.

In step S105, the power source controller 31 determines whether or not the charging of the battery 33 is completed. If the charging of the battery 33 is completed (“YES” in step S105), the processing proceeds to step S106. If the charging of the battery 33 is not completed (“NO” in step S105), the processing of step S105 is repeated. For example, on the basis of the measurement result of the voltage across the terminals of the battery 33, a determination can be made as to whether or not the charging of the battery 33 is completed.

In step S106, the power source controller 31 turns off the second switch 312 and turns on the third switch 313. The second current path P2 is cut off and the third current path P3 is enabled, so that power can be supplied from the battery 33 to the apparatus body 10A (standby state).

In the present embodiment, substantial charging of the diagnostic ultrasound apparatus 10 is completed due to the completion of the charging of the supercapacitor 32. Thus, the diagnostic ultrasound apparatus 10 can be moved to a place away from the external power source even if the charging of the battery 33 is not completed. The charging time of the supercapacitor 32 is short. Thus, the time restriction due to the charging can be shortened, and the convenience of the diagnostic ultrasound apparatus 10 is remarkably improved. Further, power stored in the supercapacitor 32 is transferred to the battery 33, and the power is supplied from the battery 33 to the apparatus body 10A, so that a constant output voltage can be obtained as a drive voltage.

Note that, in accordance with the flowchart of FIG. 4, power supply from the battery 33 to the apparatus body 10A can be made at the time of completion of charging of the battery 33. However, in a case where diagnosis with the diagnostic ultrasound apparatus 10 starts during charging of the battery 33, charging from the supercapacitor 32 to the battery 33 may be interrupted, and power may be supplied from the battery 33 to the apparatus body 10A.

Further, in a case where, with the external power source connected to the body power source 30, diagnosis with the diagnostic ultrasound apparatus 10 starts, the power source controller 31 may perform control such that power is supplied from the external power source to the apparatus body 10A.

Furthermore, until the body power source 30 is disconnected from the external power source after completion of charging of the supercapacitor 32, the battery 33 may be directly charged from the external power source, and charging from the supercapacitor 32 to the battery 33 may start at the time of disconnection of the body power source 30 from the external power source.

### Second Embodiment

A diagnostic ultrasound apparatus 10 according to a second embodiment can supply power to an apparatus body 10A while charging a battery 33 from a supercapacitor 32. That is, the diagnostic ultrasound apparatus 10 of the second embodiment is different from that of the first embodiment in that it is pass-through compatible. The configuration other than the pass-through related configuration is similar to that of the first embodiment, and thus the description thereof is not given.

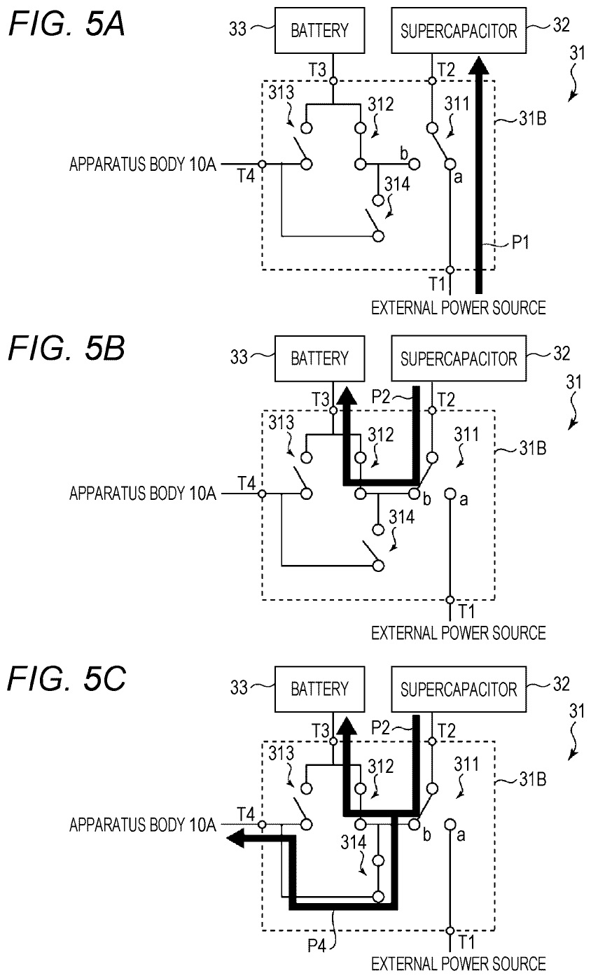

FIGS. 5A to 5C are diagrams of an exemplary electric circuit as a constituent of a power source controller 31 according to the second embodiment.

As illustrated in FIGS. 5A to 5C, the power source controller 31 includes a switching circuit 31B. The switching circuit 31B includes a fourth switch 314 in addition to first to third switches 311 to 313.

The fourth switch 314 can switch a current path, connected to an electrical contact T2 with the supercapacitor 32, to a fourth current path P4. The fourth current path P4 electrically connects the electrical contact T2 with the supercapacitor 32 and an electrical contact T4 with the apparatus body 10A (see FIG. 5C).

As illustrated in FIG. 5A, in response to connection of the first switch 311 to the a-side, a first current path P1 is enabled and the supercapacitor 32 can be charged from an external power source. As illustrated in FIG. 5B, when the second switch 312 is turned on in response to connection of the first switch 311 to the b-side, a second current path P2 is enabled and charging (power transfer) from the supercapacitor 32 to the battery 33 can be made.

As illustrated in FIG. 5C, when the fourth switch 314 is turned on with the second switch 312 kept on, the fourth current path P4 is enabled in addition to the second current path P2, and power can be supplied from the supercapacitor 32 to the apparatus body 10A while charging of the battery 33 is continued.

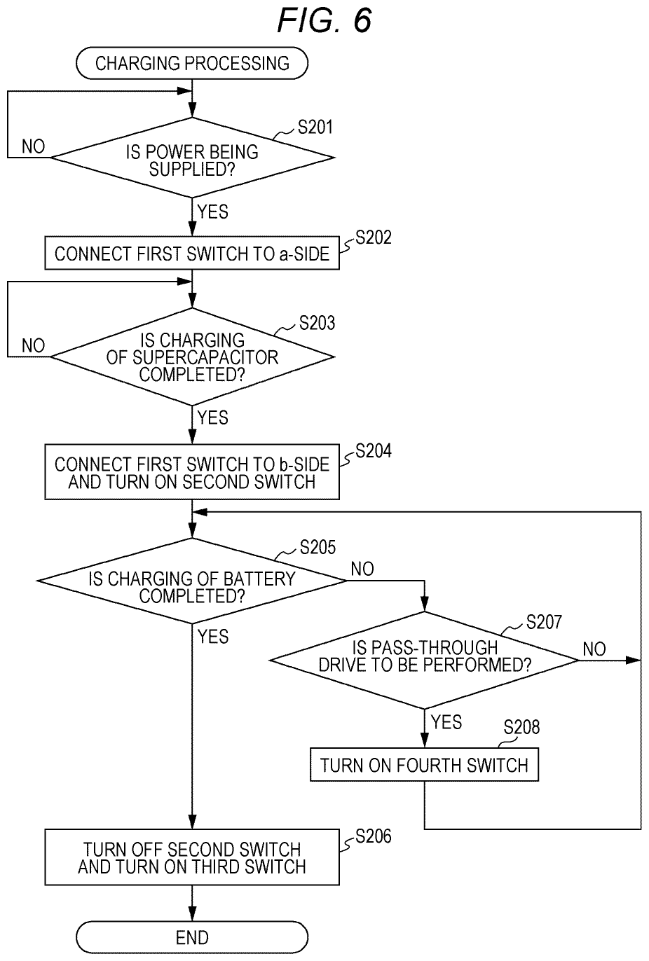

FIG. 6 is a flowchart of exemplary power source control processing according to the second embodiment. Steps S201 to S206 in FIG. 6 are similar to steps S101 to S106 in FIG. 4, and thus the description thereof is not given,

In the second embodiment, after charging of the battery 33 from the supercapacitor 32 starts, in step S207, the power source controller 31 determines whether or not to perform pass-through drive, namely, whether or not diagnosis with the diagnostic ultrasound apparatus 10 starts during charging of the battery 33.

If pass-through drive is to be performed (“YES” in step S207), the processing proceeds to step S208. If pass-through drive is not to be performed (“NO” in step S207), the processing returns to step S205, and the charging of the battery 33 is continued.

In step S208, the power source controller 31 turns on the fourth switch 314. The fourth current path P4 is enabled together with the second current path P2, and power supply from the supercapacitor 32 to the apparatus body 10A starts.

Note that, in a case where the charging of the battery 33 is completed during the pass-through drive, the second switch 312 may be switched on and the third switch may be switched off in step S206, and power supply to the apparatus body 10A may be performed through a third current path P3.

In the first embodiment, it is mainly assumed that power is supplied from the battery 33 to the apparatus body 10A. Thus, it needs to wait until the battery 33 is sufficiently charged. On the other hand, pass-through is compatible in the second embodiment. In response to start of diagnosis with the diagnostic ultrasound apparatus 10 during charging of the battery 33, power is directly supplied from the supercapacitor 32 to the apparatus body 10A.

Therefore, diagnosis can start regardless of the amount of charge of the battery 33, resulting in further improvement of the convenience of the diagnostic ultrasound apparatus 10.

### Modifications

In the first embodiment and the second embodiment, it is assumed that the apparatus body 10A is driven mainly by power supply from either the battery 33 or the supercapacitor 32. The apparatus body 10A, however, may be driven with both the supercapacitor 32 and the battery 33 as a power source.

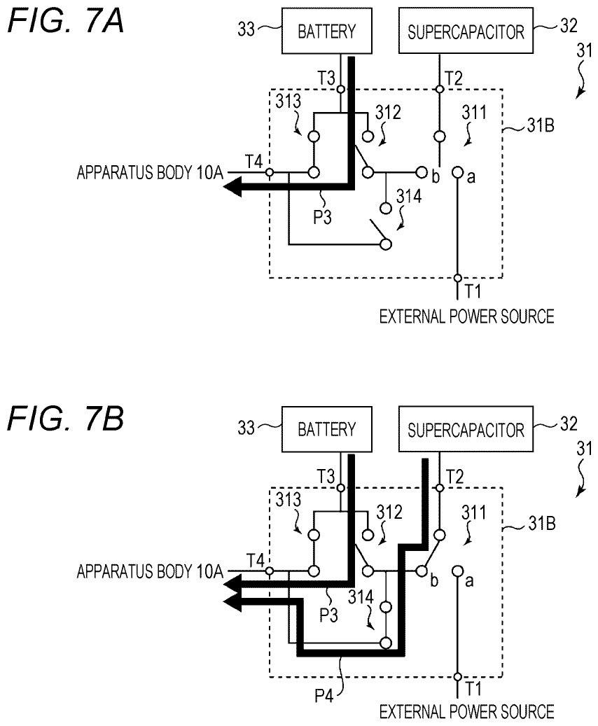

For example, as illustrated in FIG. 7A, when power is being supplied from a battery 33 to an apparatus body 10A through a third current path P3, as illustrated in FIG. 7B, turning on a fourth switch 314 enables a fourth current path P4 from a supercapacitor 32 to the apparatus body 10A in addition to the third current path P3. As a result, assist drive of a diagnostic ultrasound apparatus 10 with both the supercapacitor 32 and the battery 33 as a power source can be made.

In order to perform assist drive, power needs to remain in the supercapacitor 32 to such an extent that the assist drive can be performed. For example, in a case where the supercapacitor 32 has enough capacitance to fully charge the battery 33, power remains in the supercapacitor 32 after completion of charging of the battery 33. Further, for example, in a case where a body power source 30 is connected to an external power source at the time of completion of charging from supercapacitor 32 to the battery 33, a first switch 311 may be connected to the a-side to charge the supercapacitor 32 again. Furthermore, charging from the supercapacitor 32 to the battery 33 may be completed in a state that power to the extent that assist drive can be performed is left in the supercapacitor 32.

Still furthermore, in a case where the supercapacitor 32 has residual power that is small and below a predetermined threshold, power may be transferred from the battery 33 to the supercapacitor 32.

In assist drive, the level of power supplied to the apparatus body 10A can be increased as compared with the level of power supplied by the battery 33 alone. Therefore, the diagnostic ultrasound apparatus 10 is compatible with a diagnosis mode with large power consumption. Examples of the diagnosis mode with large power consumption include a mode for performing synthetic aperture processing. color flow mapping processing, share wave elastography, and plane wave transmission.

Further, because the residual power in the supercapacitor 32 decreases as the battery 33 is charged, it is preferable that power is efficiently extracted from the supercapacitor 32 in order to support the assist drive.

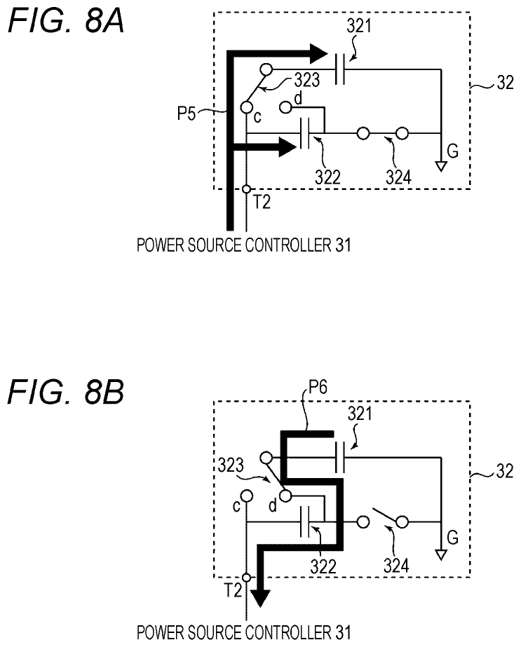

FIGS. 8A and 8B are diagrams of an exemplary electric circuit of the supercapacitor 32 suitable for assist drive.

As illustrated in FIG. 8A and 8B, the supercapacitor 32 includes, for example, two supercapacitors 321 and 322 and two switches 323 and 324. Note that the electric circuits illustrated in FIGS. 8A and 8B are examples. Thus, any electric circuit may be adopted, provided that a function can be achieved as the supercapacitor 32.

The switches 323 and 324 can switch a current path connected to an electrical contact T2, to a fifth current path P5 or a sixth current path P6. In the fifth current path P5, the supercapacitors 321 and 322 are connected in parallel between the electrical contact T2 and a ground point G (see FIG. 8A). In the sixth current path P6, the supercapacitors 321 and 322 are connected in series between the electrical contact T2 and the ground point G (see FIG. 8B).

As illustrated in FIG. 8A, when the switch 324 is turned on in response to connection of the switch 323 to the c-side, the fifth current path P5 is enabled. and the supercapacitors 321 and 322 can be simultaneously charged from an external power source.

As illustrated in FIG. 8B, when the switch 324 is turned off in response to connection of the switch 323 to the d-side, the sixth current path P6 is enabled, and power can be efficiently extracted from the supercapacitors 321 and 322.

As described above, the diagnostic ultrasound apparatuses 10 (rechargeable portable ultrasound instrument) according to the embodiments and the modifications each include, singly, a feature below or each include features below in appropriate combination.

That is, a diagnostic ultrasound apparatus 10 is a rechargeable portable ultrasound instrument for transmitting an ultrasound wave to a subject and receiving the ultrasound wave from the subject for ultrasound diagnosis, the diagnostic ultrasound apparatus 10 including: at least two chargers different in type. the chargers including a supercapacitor 32 and a battery 33; and a power source controller 31 that controls a charge operation to each of the two chargers and a power supply operation from each of the chargers to an apparatus body 10A of the diagnostic ultrasound apparatus 10. The power source controller 31 controls the charge operation and the power supply operation in accordance with the type of each of the chargers as a charge target or a power supply target.

Further, the power source control method according to an embodiment is a power source control method for a diagnostic ultrasound apparatus 10 (rechargeable portable ultrasound instrument) for transmitting an ultrasound wave to a subject and receiving the ultrasound wave from the subject for ultrasound diagnosis, the diagnostic ultrasound apparatus 10 including at least two chargers different in type, the chargers including a supercapacitor 32 and a battery 33, the power source control method including: charging each of the two chargers: and supplying power from each of the chargers to an apparatus body 10A of the diagnostic ultrasound apparatus 10. The charging and the supplying are performed in accordance with the type of each of the chargers as a charge target or a power supply target.

Furthermore, the power source control program according to an embodiment is a program for causing a power source controller 31 of a diagnostic ultrasound apparatus 10 (a controller of a rechargeable portable ultrasound instrument) to perform predetermined processing. the diagnostic ultrasound apparatus 10 being for transmitting an ultrasound wave to a subject and receiving the ultrasound wave from the subject for ultrasound diagnosis, the diagnostic ultrasound apparatus 10 including at least two chargers different in type, the chargers including a supercapacitor 32 and a battery 33, the predetermined processing including: charging each of the two chargers; and supplying power from each of the chargers to an apparatus body 10A of the diagnostic ultrasound apparatus 10. The charging and the supplying are performed in accordance with the type of each of the chargers as a charge target or a power supply target.

The power source control program can be provided through, for example, a compute readable transportable storage medium (including, for example, an optical disk, a magneto-optical disk, and a memory card) storing the power source control program. Alternatively, for example, the power source control program can be provided by downloading from a server holding the power source control program through a network.

With a diagnostic ultrasound apparatus 10 (rechargeable portable ultrasound instrument), a power source control method, and a program according to an embodiment, substantial charging of the diagnostic ultrasound apparatus 10 is completed due to completion of charging of the supercapacitor 32. Thus, the diagnostic ultrasound apparatus 10 can be moved to a place away from an external power source even if charging of the battery 33 is not completed. The charging time of the supercapacitor 32 is short. Thus, the time restriction due to the charging can be shortened, and the convenience of the diagnostic ultrasound apparatus 10 is remarkably improved. Further, power stored in the supercapacitor 32 is transferred to the battery 33, and the power is supplied from the battery 33 to the apparatus body 10A, so that a constant output voltage can be obtained as a drive voltage.

Furthermore, in the diagnostic ultrasound apparatus 10 (rechargeable portable ultrasound instrument), after the external power source charges the supercapacitor 32, the power source controller 31 performs control such that the power charged in the supercapacitor 32 is transferred to the battery 33. With this arrangement, the disadvantage of the supercapacitor 32 in that power is lowered by the self-discharge can be solved. Thus, power can be appropriately supplied to the apparatus body 10A, and the diagnostic ultrasound apparatus 10 can normally operate.

Still furthermore, in the diagnostic ultrasound apparatus 10 (rechargeable portable ultrasound instrument), the power source controller 31 performs control (pass-through drive) such that the power charged in the supercapacitor 32 is supplied to the apparatus body 10A while being transferred to the battery 33. With this arrangement, diagnosis with the diagnostic ultrasound apparatus 10 can start regardless of the amount of charge of the battery 33, resulting in further improvement of the convenience of the diagnostic ultrasound apparatus 10.

Still furthermore, in the diagnostic ultrasound apparatus 10 (rechargeable portable ultrasound instrument). in response to execution of an operation mode in which large current is consumed, the power source controller 31 performs control (assist drive) such that power from the supercapacitor 32 is added to power being supplied from the battery 33 to the apparatus body 10A. This arrangement enables supply of necessary power even in a diagnosis mode in which large power is consumed in a short time.

Still furthermore, in the diagnostic ultrasound apparatus 10 (rechargeable portable ultrasound instrument), in a case where the supercapacitor 32 has residual power below a threshold, the power source controller 31 performs control such that power is transferred from the battery 33 to the supercapacitor 32. This arrangement enables assist drive even with small residual power in the supercapacitor 32.

The present invention made by the present inventors has been specifically described above on the basis of the embodiments. The present invention, however, is not limited to the above embodiments, and thus may be modified without departing from the gist of the present invention.

For example, in a case where the ultrasound probe 20 is connectable to the diagnostic ultrasound apparatus 10 by wireless communication, the present invention is applicable to the ultrasound probe 20. That is, examples of the rechargeable portable ultrasound instrument according to the present invention includes an ultrasound probe connectable to a diagnostic ultrasound apparatus by wireless communication. In this case, the ultrasound probe 20 incorporates a function of the transmitter/receiver 12 and a function related to transmission/reception control of the body controller 11 of the diagnostic ultrasound apparatus 10. Further, the ultrasound probe 20 may have a function of the signal processor 13.

Furthermore, the diagnostic ultrasound apparatus 10 (rechargeable portable ultrasound instrument) may be provided with a notifier that notifies completion of charging during charging of the supercapacitor 32. Still furthermore, for example, the diagnostic ultrasound apparatus 10 may be provided with a notifier indicating a charging status or a power assist state from the supercapacitor 32 to the battery 33. As the notifier, for example, a light emitter such as a light-emitting diode (LED) lamp or an audio output unit such as a speaker is applicable.

Although embodiments of the present invention have been described and illustrated in detail, the disclosed embodiments are made for purposes of illustration and example only and not limitation. The scope of the present invention should be interpreted by terms of the appended claims, and is intended to include all modifications within the meaning and scope equivalent to the claims.

What is claimed is:

1. A rechargeable portable ultrasound instrument for transmitting an ultrasound wave to a subject and receiving the ultrasound wave from the subject for ultrasound diagnosis, the rechargeable portable ultrasound instrument comprising:
at least two chargers different in type, the chargers including a supercapacitor and a battery: and
a power source controller that controls a charge operation to each of the two chargers and a power supply operation from each of the chargers to an instrument body of the rechargeable portable ultrasound instrument,
wherein the power source controller controls the charge operation and the power supply operation in accordance with the type of each of the chargers as a charge target or a power supply target.

2. The rechargeable portable ultrasound instrument according to claim 1,
wherein after an external power source charges the supercapacitor. the power source controller performs control such that power charged in the supercapacitor is transferred to the battery.

3. The rechargeable portable ultrasound instrument according to claim 2,
wherein the power source controller performs control such that the power charged in the supercapacitor is supplied to the instrument body while being transferred to the battery.

4. The rechargeable portable ultrasound instrument according to claim 1.
wherein in response to execution of a diagnosis mode in which large power is consumed, the power source controller performs control such that power from the supercapacitor is added to power being supplied from the battery to the instrument body.

5. The rechargeable portable ultrasound instrument according to claim 4,
wherein in a case where the supercapacitor has residual power equal to or less than a threshold, the power source controller performs control such that power is transferred from the battery to the supercapacitor.

6. The rechargeable portable ultrasound instrument according to claim 1,
wherein the rechargeable portable ultrasound instrument serves as a diagnostic ultrasound apparatus that enables display of an ultrasound image obtained due to transmission and reception of the ultrasound wave.

7. The rechargeable portable ultrasound instrument according to claim 1,
wherein the rechargeable portable ultrasound instrument serves as an ultrasound probe connectable to a diagnostic ultrasound apparatus by wireless communication.

8. A power source control method for a rechargeable portable ultrasound instrument for transmitting an ultrasound wave to a subject and receiving the ultrasound wave from the subject for ultrasound diagnosis, the rechargeable portable ultrasound instrument including at least two chargers different in type, the chargers including a supercapacitor and a battery, the power source control method comprising:
charging each of the two chargers; and
supplying power from each of the chargers to an instrument body of the rechargeable portable ultrasound instrument,
wherein the charging and the supplying are performed in accordance with the type of each of the chargers as a charge target or a power supply target.

9. A non-transitory recording medium storing a computer readable program for causing a controller for a rechargeable portable ultrasound instrument to perform predetermined processing, the rechargeable portable ultrasound instrument being for transmitting an ultrasound wave to a subject and receiving the ultrasound wave from the subject for ultrasound diagnosis, the rechargeable portable ultrasound instrument including at least two chargers different in type, the chargers including a supercapacitor and a battery, the predetermined processing comprising:
charging each of the two chargers; and
supplying power from each of the chargers to an instrument body of the rechargeable portable ultrasound instrument,
wherein the charging and the supplying are performed in accordance with the type of each of the chargers as a charge target or a power supply target.
